(19) United States
(12) Patent Application Publication
(10) Pub. No.: US 2026/0355521 A1
(43) Pub. Date: Nov. 12, 2026

(54) SYSTEM AND METHOD FOR DETRENDING STIMULATION ARTIFACTS IN NEUROMODULATION SYSTEMS

(71) Applicant: THE JOHNS HOPKINS UNIVERSITY, Baltimore, MD (US)

(72) Inventors: William Stanley ANDERSON, Towson, MD (US); Yousef SALIMPOUR, Baltimore, MD (US); Kelly MILLS, Silver Spring, MD (US)

(21) Appl. No.: 19/111,947

(22) PCT Filed: Sep. 22, 2023
(86) PCT No.: PCT/US2023/033444

§ 371 Date: Mar. 14, 2025

Related U.S. Application Data
(60) Provisional application No. 63/377,073, filed on Sep. 26, 2022.

(51) Int. Cl. A61N 1/36; A61N 1/02

(52) U.S. Cl. CPC A61N 1/36139; A61N 1/025; A61N 1/36171

(57) ABSTRACT

A method, computer system, and a non-transitory computer-readable medium are disclosed that performs instructions including receiving, from one or more electrodes, information identifying brain activity with a stimulus-induced trend signal for a first time period; trend identification, based on the information identifying the brain activity for the first time period using wavelet-based multi-resolution signal decomposition method, estimated brain activity without the stimulus trend signal for a second time period that is to occur after the first time period; inserting the detrended brain activity into the information for a second time period; determining, based on the detrended brain activity for the second time period, a brain stimulus for the second time period; and causing the brain stimulus to be applied the second time period. The presented method could be implemented at a hardware level and be applied to variety of neuromodulation techniques induces trend to the recoded data after brain stimulation.

### CROSS REFERENCE TO RELATED APPLICATIONS

This is the national stage entry of International Patent Application No. PCT/US 2023/033444, filed on Sep. 22, 2023, and published as WO 2024/072696 A 1 on Apr. 4, 2024, which claims the benefit of U.S. Provisional Patent Application No. 63/377,073, filed on Sep. 26, 2022, which are hereby incorporated by reference in their entireties.

### FIELD

This application is directed to neuromodulation systems, and in particular, to systems and methods for removing trends due to stimulation-induced artifacts in neuromodulation systems.

### BACKGROUND

Brain stimulation may be used to alter, enhance, and/or improve brain function in neurological disorders, such as epilepsy, movement disorders, psychiatric disorders, and/or the like. A control device may stimulate a brain of a patient by causing electrical stimulation pulses to be applied to the brain using a set of electrodes. The control device may trigger the electrodes to provide a set of electrical stimulation pulses, which may cause brain function to be altered, enhanced, and/or improved. The set of electrodes may be disposed of on the patient's brain surface, or within the brain substance and may be connected to the control device via a set of wires.

Neuromodulation is a rapidly expanding area of translational neuroscience that involves stimulation, excitation, inhibition, and alteration of activity in the nervous system using electrical, electromagnetic, chemical, and even mechanical stimuli. The use of electrophysiological recordings has widely increased in modern neuromodulation technologies, specifically in closed-loop neuromodulation applications to increase the efficacy of neuromodulation based clinical treatments. Currently, a variety of neuromodulation techniques are being utilized for the treatment of neurological disorders such as Parkinson's disease and other movement disorders, chronic pain, psychiatric disorders, epilepsy, and many others. Electrical neuromodulation represents electrical or electromechanical stimulation of the nervous system in various structures such as the brain, spinal cord, and peripheral nerves.

The use of electrophysiological features as biofeedback in neuromodulation which is known as closed-loop neuromodulation, for adjusting the applied therapy characteristics is increasingly being used. Brain stimulation-related artifacts such as trend artifacts are one of the main challenges in closed-loop neuromodulation systems in measuring the results of simulation and using it as biofeedback to adjust the stimulation parameters. In order to have an effective neuromodulation treatment, the system must be able to record and stimulate simultaneously during regular neural activities. In clinical applications, neuromodulation techniques rely mainly on directly applied electrical stimulation. Electrical neuromodulation consists of applied electrical stimulation of the nervous system in various structures such as the brain, spinal cord, and peripheral nerves. The use of electrophysiological features as biofeedback in neuromodulation techniques for adjusting the stimulation parameters such as intensity/amplitude, frequency, and the neuroanatomical target is widely increasing. To have an effective neuromodulation treatment, the system must be able to record and stimulate simultaneously during regular neural activities. However, the persistent stimulation artifacts in the form of rapid changes and also in the form of slow trends that distort the recorded signals represent a severe challenge and so an effective artifact reduction technique is needed. Although, some techniques have already been introduced to account for the rapid transient effects, changes in the trend of the signal as a result of charge accumulation are still one of the main challenges in the stimulus artifact removal process. Therefore, persistent stimulation artifacts that distort recorded signals represent a severe challenge and so an effective artifact reduction technique is needed.

### SUMMARY

According to examples of the present disclosure, a method is disclosed that comprises receiving, from one or more electrodes, information identifying brain activity with a stimulus trend artifact signal for a first time period; detecting, based on the information identifying the brain activity for the first time period, a trend in the stimulus trend artifact for the first time period and detrending the brain activity without the stimulus trend artifact signal for a second time period that is to occur after the first time period; inserting the estimated brain activity without the stimulus trend artifact signal into the information for a second time period; determining, based on the estimated brain activity for the second time period, a brain stimulus for the second time period; and causing the brain stimulus to be applied the second time period or the detrending data based on the estimated brain activity can be used for later analysis or patient treatment management.

According to examples of the present disclosure, a computer system is disclosed that comprises a hardware processor; a non-transitory computer readable medium that stores instructions that when executed by the hardware processor perform a method comprising: receiving, from one or more electrodes, information identifying brain activity with a stimulus trend artifact signal for a first time period; detecting, based on the information identifying the brain activity for the first time period, a trend in the stimulus trend artifact for the first time period and detrending the brain activity without the stimulus trend artifact signal for a second time period that is to occur after the first time period; inserting the estimated brain activity without the stimulus trend artifact signal into the information for a second time period; determining, based on the estimated brain activity for the second time period, a brain stimulus for the second time period; and causing the brain stimulus to be applied the second time period.

According to examples of the present disclosure, a non-transitory computer readable medium that stores instructions that when executed by a hardware processor perform a method, comprising: receiving, from one or more electrodes, information identifying brain activity with a stimulus trend artifact signal for a first time period; detecting, based on the information identifying the brain activity for the first time period, a trend in the stimulus trend artifact for the first time period and detrending the brain activity without the stimulus trend artifact signal for a second time period that is to occur after the first time period; inserting the estimated brain activity without the stimulus trend artifact signal into the information for a second time period; determining, based on the estimated brain activity for the second time period, a brain stimulus for the second time period; and causing the brain stimulus to be applied the second time period.

Various additional features can be included in the method, the computer system, and the non-transitory computer readable medium including one or more of the following features. The brain stimulus is associated with a frequency and a phase determined based on the estimated brain activity for the second time period. The method further comprises determining the stimulus trend artifact signal during the first time period associated with a prior brain stimulus; and determining a trend artifact-removed brain activity for the first time period based on the trend artifact. Estimating the brain activity for the second time period further comprises: estimating the brain activity for the second time period based on the trend artifact-removed brain activity. The brain stimulus is caused to occur during a period of rhythmic brain activity. Estimating the brain activity for the second time period further comprises: estimating the brain activity using a parametric spectral estimation technique for modeling band limited oscillations.

### BRIEF DESCRIPTION OF THE DRAWINGS

The accompanying drawings, which are incorporated in and constitute a part of this specification, illustrate embodiments of the present teachings and, together with the description, serve to explain the principles of the present teachings. In the figures:

FIG. 1 shows a system diagram according to examples of the present disclosure;

FIG. 2 shows an example environment in which systems and/or methods described herein may be implemented;

FIG. 3 shows a first example of a recording/prediction diagram according to examples of the present disclosure;

FIG. 4 shows an example of a trend in electrophysiology recording and illustrates its dissociation with artifacts such as stimulation artifacts;

FIG. 5 shows how conventional detrending such as the linear detrending system, fails in capturing nonlinear trends in an illustrative example;

FIG. 6 shows the performance of the present method on the same illustrative example from FIG. 5;

FIG. 7 shows an example of the nonlinear trend in neuronal recording data;

FIG. 8 Illustrates the idea of nonlinear detrending and its association with the wavelet-based denoising method;

FIG. 9 shows the decomposition tree for finding the best branch for describing the nonlinear trend in data;

FIG. 10 shows the output of each branch in the decomposition tree for selecting the trend of data is shown. Depending on the characteristics of the signal and also the expectation from the detrending process, one of the lower branches in the decomposition tree;

FIG. 11 shows the reconstruction tree, the selected branch with the trend data is kept from the decomposition process and the rest of data set to zero;

FIG. 12 shows the entire wavelet base filter bank consist of decomposition and reconstruction stages;

FIG. 13 shows a representative data regarding the difference between output of the nonlinear detrending method proposed here and the bandpass in the same frequency range;

FIG. 14 shows illustrative data before and after detrending using the present method;

FIG. 15 shows the time-frequency distribution of the original data from FIG. 14;

FIG. 16 shows the time-frequency distribution of the illustrative data after detrending;

FIG. 17 shows a method for artifact removal for neuromodulation systems according to examples of the present disclosure; and

FIG. 18 illustrates a schematic view of a computing system according to examples of the present disclosure.

### DETAILED DESCRIPTION

Reference will now be made in detail to embodiments, examples of which are illustrated in the accompanying drawings and figures. In the following detailed description, numerous specific details are set forth in order to provide a thorough understanding of the invention. However, it will be apparent to one of ordinary skill in the art that the invention may be practiced without these specific details. In other instances, well-known methods, procedures, components, circuits and networks have not been described in detail so as not to unnecessarily obscure aspects of the embodiments.

Electrical pulse-based brain stimulation may be performed using a fixed set of electrical pulses applied to a brain of a patient for a period of time. This technique may result in alteration, improvement, enhancement, and/or the like to brain function of the brain of the patient. However, using a constant set of electrical pulses may be inefficient, resulting in wasted energy resources, which may hinder miniaturization of brain stimulation devices. Moreover, using a fixed set of electrical pulses may result in relatively poor clinical outcomes. For example, using constant stimulation may result in lower thresholds for stimulation-associated side effects (e.g., as a result of activating or inhibiting structures proximate to locations at which the constant stimulation is applied).

Generally speaking, an adaptive trend removal system and method for removing artifactual trends in recorded electrophysiology data after brain stimulation from the same or neighboring recording sites are disclosed. For each recording channel that is used, two parallel computational blocks are considered, the first one for transient artifact removal, and the second one for detrending the signal for specified time intervals using a wavelet based multiresolution signal analysis technique. In order to detrend the recorded after-stimulus signal, the time interval is set manually based on baseline recording assessments. The discrete wavelet transform based multiresolution signal decomposition is then applied to a segment of the after-stimulus signal. This generates the coefficients of multiple time scales of approximation and detail. The approximation signal from the last stage of decomposition is considered as a trend, and all other coefficients are set to zero, and the artifactually altered signal is reconstructed. This reconstructed signal is considered a trend and is subtracted from the original signal. This method can be fully implemented on a system-on-chip (SoC) technology and easily added to existing neuromodulation systems. It can also be used for both offline and online signal detrending. The present adaptive, multiresolution signal decomposition-based detrending system and method have the potential for use in a wide range of brain stimulation techniques utilizing closed-loop neuromodulation methods.

For example, a device may measure brain activity, reduce and/or remove artifact trends, dynamically identify a stimulus pulse to control the estimated future brain activity and cause the stimulus pulse to be applied to alter future brain activity. Moreover, based on reducing the utilization of energy resources, some implementations described herein enable miniaturization and/or implantability of a device to perform phase-dependent stimulation with stimulus artifact reduction, or other forms of closed-loop neuromodulation with stimulation artifact reduction. This technique may be applicable in treatment relating to Parkinson's disease, theta rhythm issues relating to memory, Schizophrenia, Alzheimer's disease, and/or the like. The systems/methods described herein have the full flexibility to adapt to a variety of neuromodulation systems with the potential to combine with typical neuromodulation techniques, including Transcranial Electrical Stimulation (TES), Transcranial Magnetic Stimulation (TMS), Deep Brain Stimulation (DBS), Direct Cortical Stimulation (DCS), and Ultrasound therapies (US).

Trends due to stimulus-induced artifacts distort the electrophysiological signal, alter feature detection, and significantly change modeling parameter estimation. Trend artifact removal can increase the accuracy of brain recordings and provide a more representative view of actual brain network behavior. Here, an example of stimulus trend artifact removal using the estimated signal from a predictive model is illustrated. The start time is synchronized with the stimulation trigger, and the stop time is estimated based on the stimulus artifact duration. To eliminate the trend stimulus artifact from the recordings, an optimized wavelet based multiresolution signal decomposition based method can be used to detect nonlinear trend in the recorded signal during and after stimulation events.

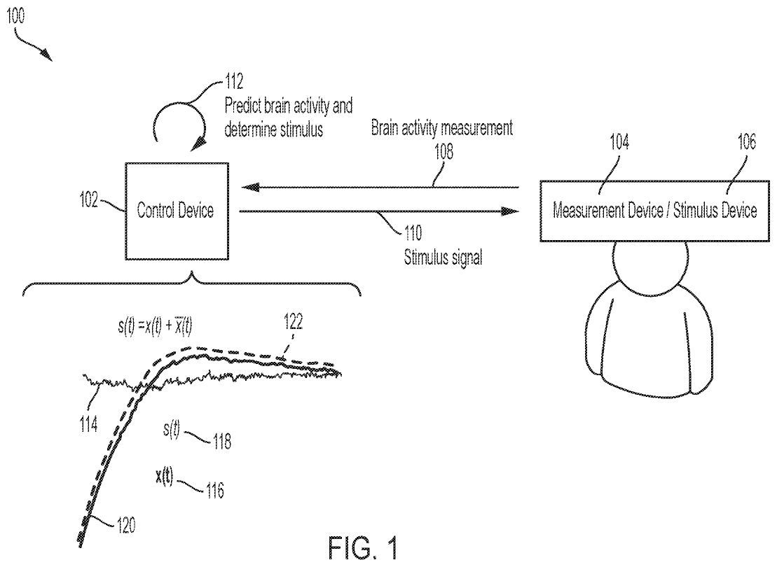

FIG. 1 shows a system diagram according to examples of the present disclosure. As shown in FIG. 1, example implementation 100 may include control device 102 that communicates with measurement device 104 and/or stimulus device 106. In some implementations, control device 102 or another device may wirelessly power measurement device 104 and/or stimulus device 106 via a wireless power transmission functionality.

As further shown in FIG. 1, and by reference number 108, control device 102 may receive information identifying a brain activity measurement from measurement device 104 and use it as a biofeedback for closed-loop brain stimulation. For example, control device 102 may receive information identifying a phase, amplitude, frequency, and/or the like of brain activity of a brain of a patient. In some implementations, control device 102 may receive information identifying brain activity for a particular time interval. In some implementations, control device 102 may determine whether a threshold level of brain activity is detected for the particular time interval. For example, control device 102 may determine that a threshold level of beta activity (e.g., activity in a range of 13 Hertz (Hz) to 30 Hz) is detected. Additionally, or alternatively, control device 102 may determine that a threshold level of phase-amplitude coupling is detected. In this case, control device 102 may use a rolling dynamic phase amplitude coupling (PAC) estimation technique to determine the phase amplitude coupling in intervals of less than or equal to 1 second, 500 milliseconds, and/or the like. In this way, control device 102 may avoid performing complex calculations to predict brain activity when the brain activity is below a threshold for which corrective stimuli are to be applied, thereby reducing processor utilization, improving battery life, improving a lifespan of example implementation 100, and/or the like.

As further shown in FIG. 1, and by reference number 112, control device 102 may predict brain activity and determine a stimulus to apply to the brain of the patient. For example, and as shown by reference number 114, based on information identifying brain activity for a first time period, control device 102 may predict brain activity for a second time period occurring after the first time period. Reference number 114 is an example of a recorded brainwave signal from a motor cortex of a patient, which comprises a first segment 116 representative of a stimulus signal with the trend and denoted by x(t), a second segment 118 representative of a recorded brainwave signal after detrending and denoted by s(t), and a third segment 120 representative by a nonlinear trend signal with stimulus artifact and denoted by x̅(t), which are related by the following expression:

s(t)=x(t)−x̅(t)

where the portion of third segment 10 shown in box 112 in greater detail. In this case, control device 102 may determine a set of electrical pulses to correspond to a predicted period of rhythmic brain activity. For example, control device 102 may determine the set of electric pulses based on signal predictive modeling of rhythmic activity with forward-prediction to time the set of electric pulses in accordance with the rhythmic activity. Some examples may include using auto-regressive modeling, generalized linear modeling, machine learning-based modeling, and/or the like. In this way, control device 102 enables electrical pulse-based brain stimulation using reduced power and with improved efficacy relative to a constant set of brain rhythmic activities.

The trend in the brain recording data is mostly related to the interaction between brain tissue and the stimulator system, such as electric charge build-up in the stimulation site due to the capacitive interaction between metallic contact and the brain tissue. The application of the disclosed system and method for removing nonlinear trends from brain recording is not limited to the specific type of brain stimulation such as phase-dependent stimulation and it has the potential to generalize to any neuromodulation technique that involves induction of trend in the neural recording data after brain stimulation.

Control device 102 removes one or more artifacts from measured brain activity when determining estimated brain activity. For example, control device 102 may identify one or more artifacts including trends in brain activity during a first time period corresponding to one or more electrical pulses provided during the first time period, and may remove the one or more artifacts in the brain activity to determine baseline brain activity. In some implementations, control device 102 may detect the trend using wavelet based multiresolution signal decomposition to interpolate brain activity during periods when artifacts occur as a result of application of timed stimulus pulses. In this case, control device 102 may detect subsequent brain activity based on the baseline brain activity, thereby improving accuracy of a subsequent brain activity detection relative to detecting with the artifacts included. In some implementations, control device 102 may use a wavelet based nonlinear trend estimation technique to predict brain activity. For example, control device 102 may detect band limited oscillations in brain activity using the nonlinear trend estimation technique, and may estimate subsequent brain activity based on detrended band limited oscillations in brain activity. In some implementations, control device 102 may apply a multiresolution signal decomposition technique to detect brain activity.

As further shown in FIG. 1, and by reference number 110, control device 102 may provide a stimulus signal to stimulus device 106. For example, control device 102 may cause stimulus device 106 to provide a set of electrical pulses to the brain of the patient during the predicted period of rhythmic brain activity. In this way, control device 102 enables closed-loop neuromodulation based on detected brain signals.

In some implementations, the set of electrical pulses may be timed in accordance with a phase of brain activity (e.g., within a particular band of oscillatory frequencies), a frequency of brain activity, and/or the like predicted based on past brain activity. In some implementations, control device 102 may cause stimulus device 106 to provide a variable-pulse stimulus. For example, control device 102 may cause electrical pulses to vary in frequency, phase, intensity, and/or the like, thereby enabling reduced power utilization, improved efficacy, and/or the like relative to a constant set of electrical pulses.

As indicated above, FIG. 1 is provided merely as one or more examples. Other examples may differ from what is described with regard to FIG. 1.

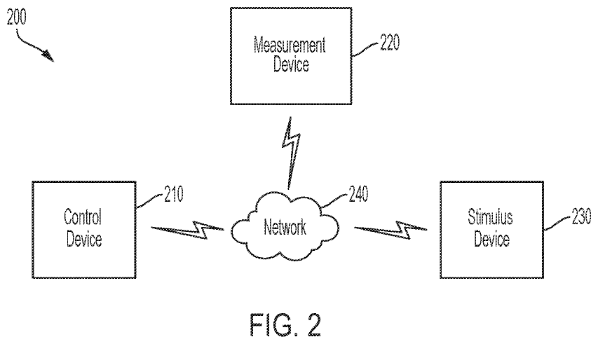

FIG. 2 is a diagram of an example environment 200 in which systems and/or methods described herein may be implemented. As shown in FIG. 2, environment 200 may include a control device 210, a measurement device 220, a stimulus device 230, and a network 240. Devices of environment 200 may interconnect via wired connections, wireless connections, or a combination of wired and wireless connections (e.g., for power transmission, data transmission, and/or the like).

Control device 210 includes one or more devices capable of receiving, generating, storing, processing, and/or providing information associated with phase-dependent or other forms of neuromodulation requiring stimulation artifact and trend reduction. For example, control device 210 may include a communication and/or computing device, such as a computer (e.g., a desktop computer, a laptop computer, a tablet computer, a handheld computer), a medical device, a wearable communication device (e.g., a smart wristwatch, a pair of smart eyeglasses, a wearable medical device, an implantable medical device, etc.), or a similar type of device. In some implementations, control device 210 may be an external device connected to measurement device 220 and/or stimulus device 230. In some implementations, control device 210, measurement device 220, and stimulus device 230 may be an integrated system-on-chip device that is at least partially implanted into a patient.

Measurement device 220 includes one or more devices capable of receiving, generating, storing, processing, and/or providing information associated with a measurement of brain activity. For example, measurement device 220 may include an electrode (e.g., a measurement electrode) for sensing a phase, a frequency, an amplitude, cross-frequency coupling, and/or the like of brain activity of a brain of a patient. In some implementations, measurement device 220 may be a measurement device mounted onto a head of a patient, a measurement device surgically implanted into a head of a patient, and/or the like.

Stimulus device 230 includes one or more devices capable of receiving, generating, storing, processing, and/or providing information associated with stimulation of a brain. For example, stimulus device 230 may include an electrode (e.g., a stimulus electrode) or multiple electrodes for applying an electrical pulse to a brain of a patient. In some implementations, stimulus device 230 may be a stimulation device mounted onto a head of a patient, a stimulation device surgically implanted into a head of a patient, and/or the like.

Network 240 includes one or more wired and/or wireless networks. For example, network 240 may include a cellular network (e.g., a long-term evolution (LTE) network, a code division multiple access (CDMA) network, a 3G network, a 4G network, a 5G network, another type of next generation network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, a cloud computing network, a BLUETOOTH network using a BLUETOOTH communication protocol, a near-field communication network, or the like, and/or a combination of these or other types of networks. The network can also provide data security, authorization, and/or authentication using one or more public and/or private cryptographic protocols to provide a measure of protected health information assurance.

The number and arrangement of devices and networks shown in FIG. 2 are provided as one or more examples. In practice, there may be additional devices and/or networks, fewer devices and/or networks, different devices and/or networks, or differently arranged devices and/or networks than those shown in FIG. 2. Furthermore, two or more devices shown in FIG. 2 may be implemented within a single device, or a single device shown in FIG. 2 may be implemented as multiple, distributed devices. Additionally, or alternatively, a set of devices (e.g., one or more devices) of environment 200 may perform one or more functions described as being performed by another set of devices of environment 200.

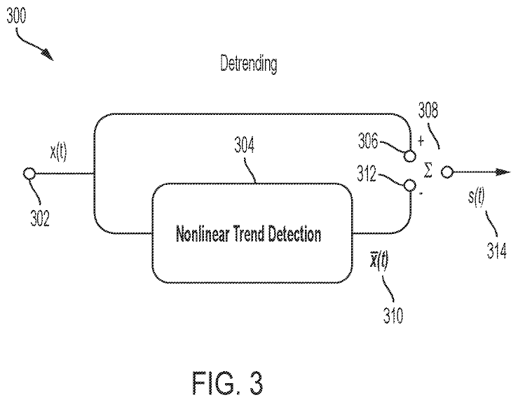

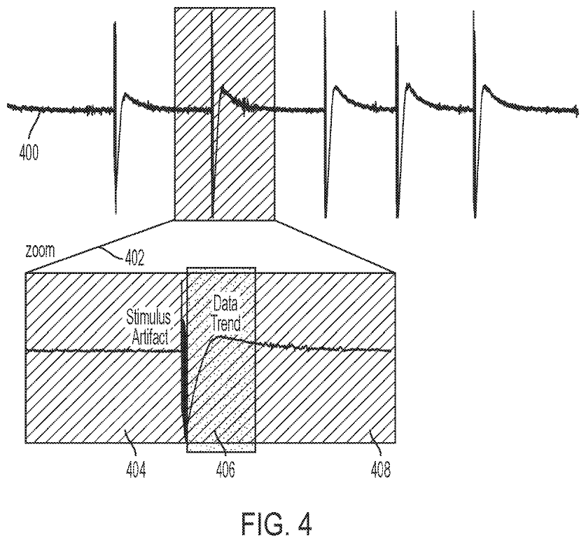

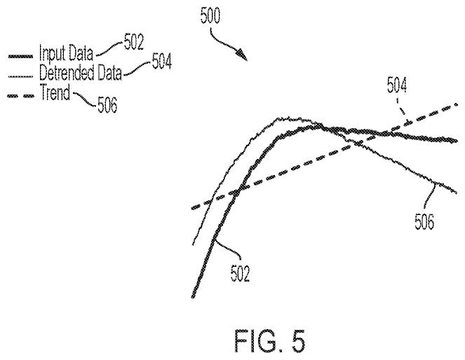

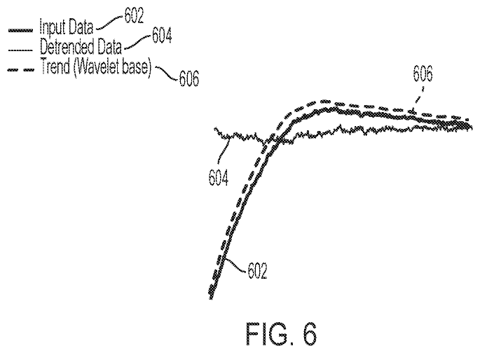

As described below in relation to FIG. 3, FIG. 4, FIG. 5, and FIG. 6, the stimulus artifact is removed by utilizing the output from the predictive model, and by activation of a switching mechanism. The estimated waveform was substituted for the stimulus artifact during active stimulation. The switching time is synchronized with the stimulation trigger, and the stop time is estimated based on the average stereotypic stimulus artifact duration. Additionally, an optimized block is provided for dynamically updating the predictive model parameters.

FIG. 3 shows a first example of a recording/prediction diagram 300 according to examples of the present disclosure. Input signal x(t) 302, which is the first segment 116, is provided to the nonlinear trend detection 304 and provided to first terminal 306 of switch 308. Output 310 from trend detection 304, denoted x(t), is provided to second terminal 312 of trend subtraction switch 308. Switch 308 outputs signal s(t) 314.

FIG. 4 shows a recorded signal 400 with an estimated signal portion according to examples of the present disclosure. In FIG. 4, a zoomed in portion 402 of the recorded signal 400 is shown segmented into a first portion 404, a second portion 406, and a third portion 408. First portion 404 and third portion 408 represent the recorded brainwave signal from the patient. Second portion 406 represents the modeled, predictive signal that has the data trend artifact removed. Second portion 406 is modeled to have smooth transitions between first portion 404 and third portion 408 that compensate for edge effects associated with the data trend removal without having any discontinuities in the beginning and end of the modeled signal. The zoomed in portion 402 of the recorded signal 400 is shown that is representative of the relationships between the first portion 404 that is representative of the recorded signal 400 with an artifact portion and the second portion 406 that is representative of the recorded signal 400 with the data trend portion that follows the stimulus artifact portion. The stimulus artifact portion in the first portion 404 shows a spike in the recorded signal 400 that is followed by a sharply decreasing signal in the data trend portion of the second portion 406.

FIG. 5 shows stimulus artifact detrending curves 500 according to examples of the present disclosure. Recorded signal representative of an input data signal 502 is shown. A modeled trend line or curve 504 is shown that is representative of the sloping curve of the input data signal 502. A detrended data signal 506 is also shown that is representative of the input data signal 502 that has been adjusted based on the modeled trend line or curve 504.

FIG. 6 shows stimulus artifact detrending curves 600 according to examples of the present disclosure. Recorded signal representative of an input data signal 602 is shown. A modeled wavelet base trend curve 604 is shown that is representative of the sloping curve of the input data signal 602. A detrended data signal 606 is also shown that is representative of the input data signal 602 that has been adjusted based on the modeled wavelet base trend curve 604.

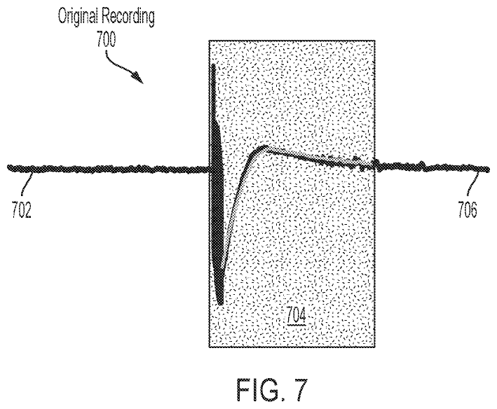

FIG. 7 shows a recorded signal 700 with a modeled estimated signal portion according to examples of the present disclosure. In FIG. 7, the recorded signal 700 is shown segmented into a first portion 702, a second portion 704, and a third portion 706. First portion 702 and third portion 706 represent the recorded brainwave signal from the patient.

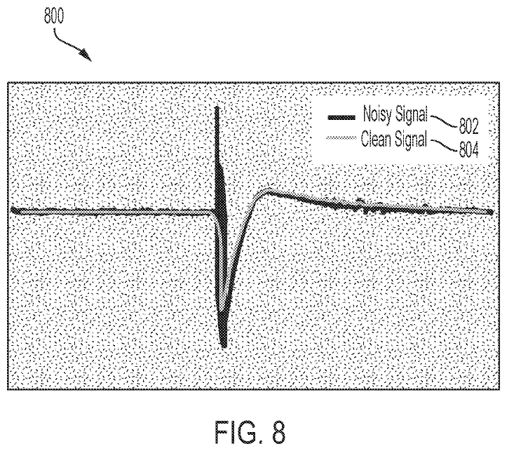

FIG. 8 shows a denoising-wavelet based signal 800 according to examples of the present disclosure. The denoising-wavelet based signal 800 is shown with a noisy signal 802 and a clean signal 804 that has been denoised using a wavelet-based approach.

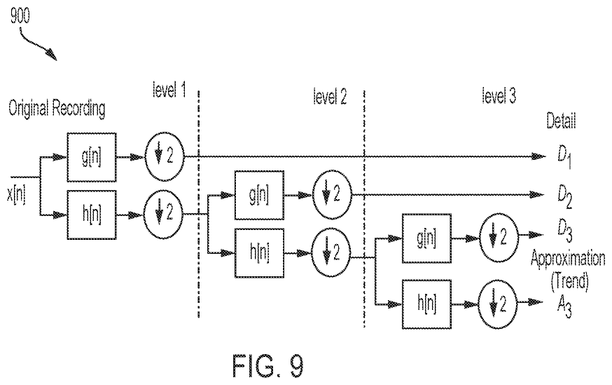

FIG. 9 shows a denoising-wavelet based approach 900 according to examples of the present disclosure. The denoising-wavelet based approach 900 comprises three processing levels, which are a first level 902, a second level 904, and a third level 906. The first level 902 obtains an input signal x[n] 908 and separates the input signal x[n] 908 into first portion g[n] 910 and a second portion h[n] 912. The second level 904 separates the second portion h[n] 912 into third portion g[n] 914 and a fourth portion h[n] 916. The third level 906 separates the fourth portion h [n] 916 into fifth portion g[n] 918 and a sixth portion h[n] 920. The third level 906 also outputs three detail coefficients d1 922, d2 924, and d3 926 and an approximation trend coefficient a1 928.

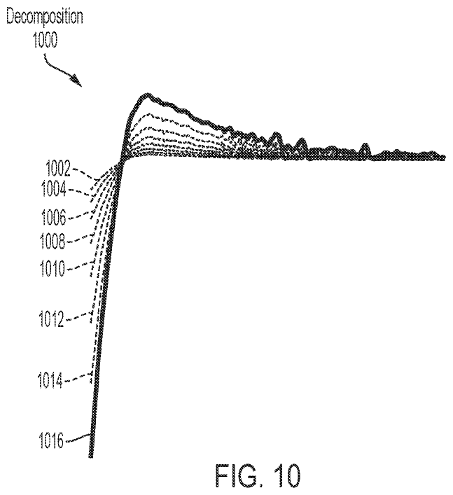

FIG. 10 shows a denoising-wavelet based decomposition signal comparison 1000 according to examples of the present disclosure. As shown in FIG. 10, an original signal 1002 is shown as compared with eight iterations, which include A1 1004, A2 1006, A3 1008, A4 1010, A5 1012, A6 1014, A7 1016, and A8 1018 of the denoising-wavelet based approach 900 of FIG. 9.

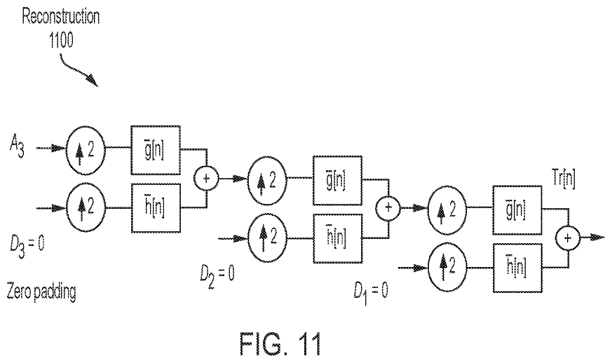

FIG. 11 shows a signal reconstruction process 1100 according to examples of the present disclosure. As shown in FIG. 11, in the reconstruction tree, only the branch with the trend is kept from the decomposition process from FIG. 9 and the rest of the branches are padded with zero. This allows the reconstruction process to reconstruct only the associated trend of the input signal.

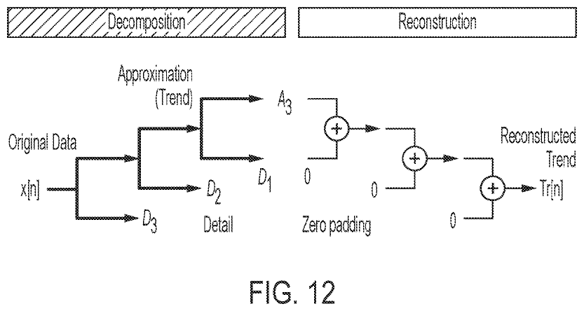

FIG. 12 shows a signal decomposition and reconstruction process 1200 according to examples of the present disclosure. As FIG. 12 shows, the wavelet base detrending filter bank consist of decomposition and reconstruction stages. For extracting the nonlinear trend, the selected branch with the trend data is kept from the decomposition process and the rest of data set to zero.

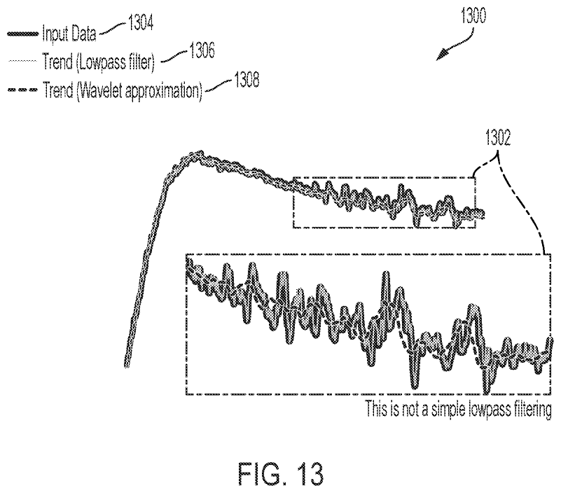

FIG. 13 shows stimulus artifact detrending curves 1300 according to examples of the present disclosure. The stimulus artifact detrending curves 1300 are shown in greater detail in the zoomed-in portion 1302. The recorded signal representative of an input data signal 1304 is shown. A modeled lowpass filter trend curve 1305 and a modeled wavelet base approximation trend curve 1308 are shown that is representative of the input data signal 1304. As this figure shows, the wavelet-based trend curve is different from the output of the low-pass filter.

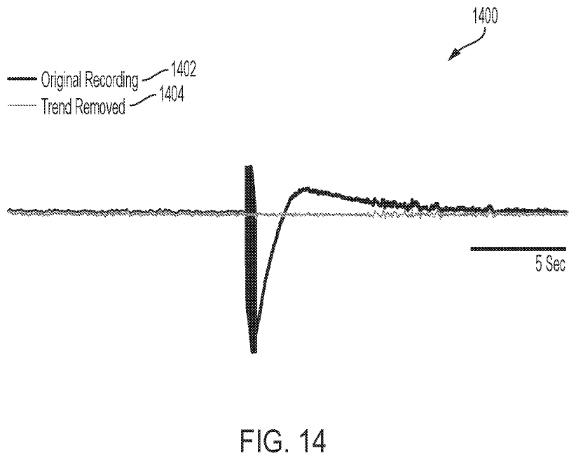

FIG. 14 shows a sample detrending curve 1400 according to examples of the present disclosure. The sample detrending curve 1400 shows an original recorded signal 1402 and a signal with the trend removed 1404 using the processes described herein.

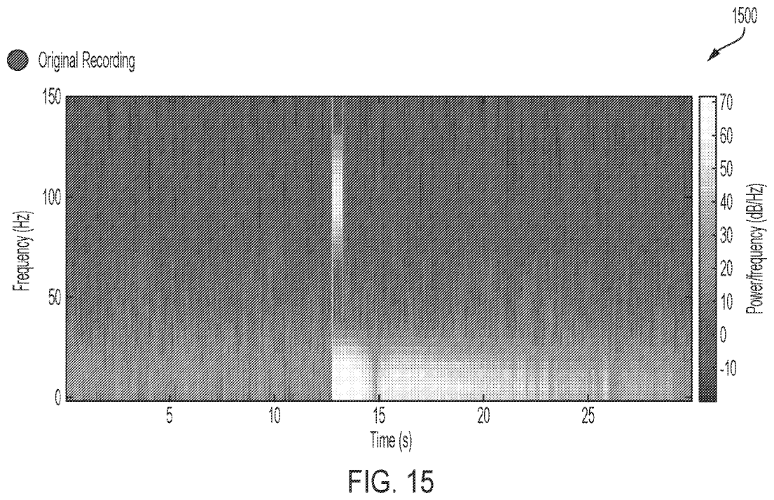

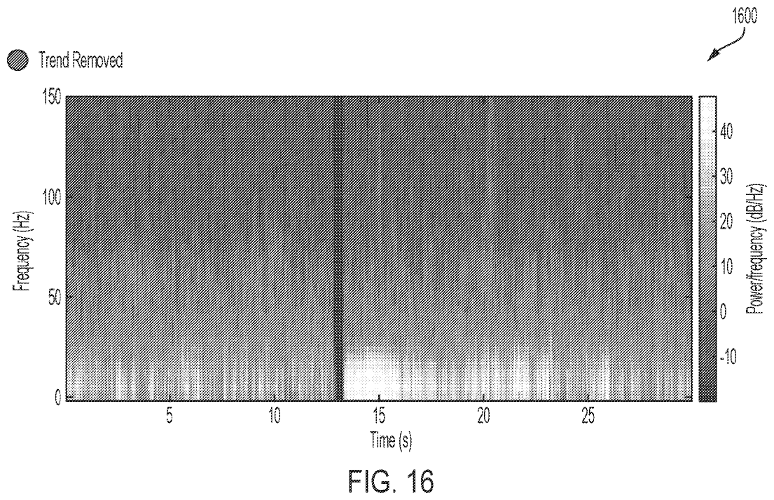

FIG. 15 shows a sample channel spectrum 1500 according to examples of the present disclosure. FIG. 16 shows a sample channel spectrum with the trend removed 1600 according to examples of the present disclosure.

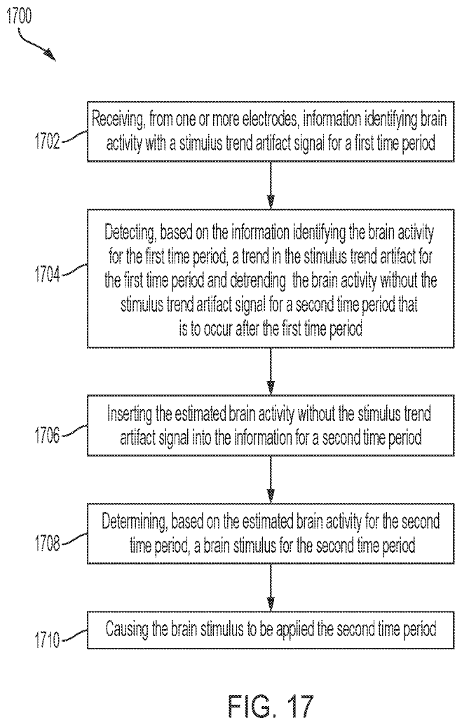

FIG. 17 shows a method for artifact removal for neuromodulation systems 1700 according to examples of the present disclosure. In some implementations, one or more process blocks of FIG. 17 may be performed by a control device (e.g., control device 210). In some implementations, one or more process blocks of FIG. 17 may be performed by another device or a group of devices separate from or including the control device, such as a measurement device (e.g., measurement device 220), a stimulus device (e.g., stimulus device 230), and/or the like.

As shown in FIG. 17, process 1700 may include receiving, from one or more electrodes, information identifying brain activity with a stimulus trend artifact for a first time period (block 1702). For example, the control device (e.g., using analysis module(s) 1802, processor(s) 1804, storage media 1806, machine learning module(s) 1808, network interface 1807, and/or the like) may receive, from one or more electrodes, information identifying brain activity for a first time period, as described above.

As further shown in FIG. 17, process 1700 may include trend detection based on the information identifying the brain activity for the first time period, detrending brain activity without the stimulus trend artifact signal for a second time period that is to occur after the first time period (block 1704). For example, the control device (e.g., using analysis module(s) 1802, processor(s) 1804, storage media 1806, machine learning module(s) 1808, network interface 1807, and/or the like) may predict, based on the information identifying the brain activity for the first time period, estimated brain activity for a second time period that is to occur after the first time period, as described above.

As further shown in FIG. 17, process 1700 may include inserting the estimated brain activity without the stimulus trend artifact into the information for the second time period, estimated (block 1706). For example, the control device (e.g., using analysis module(s) 1802, processor(s) 1804, storage media 1806, machine learning module(s) 1808, network interface 1807, and/or the like) may determine, based on the estimated brain activity for the second time period, a brain stimulus without the stimulus trend artifact for the second time period, as described above. In some implementations, the modeled brain stimulus is associated with a frequency and a phase determined based on the estimated brain activity for the second time period.

As further shown in FIG. 17, process 1700 may be included, causing the brain stimulus to be applied in the second time period (block 1708). For example, the control device (e.g., using analysis module(s) 1802, processor(s) 1804, storage media 1806, machine learning module(s) 1808, network interface 1807, and/or the like) may cause the brain stimulus to be applied in accordance with the frequency and the phase during the second time period, as described above.

Process 1700 may include additional implementations, such as any single implementation or any combination of implementations described below and/or in connection with one or more other processes described elsewhere herein.

Although FIG. 17 shows example blocks of process 1700, in some implementations, process 1700 may include additional blocks, fewer blocks, different blocks, or differently arranged blocks than those depicted in FIG. 17. Additionally, or alternatively, two or more of the blocks of process 1700 may be performed in parallel.

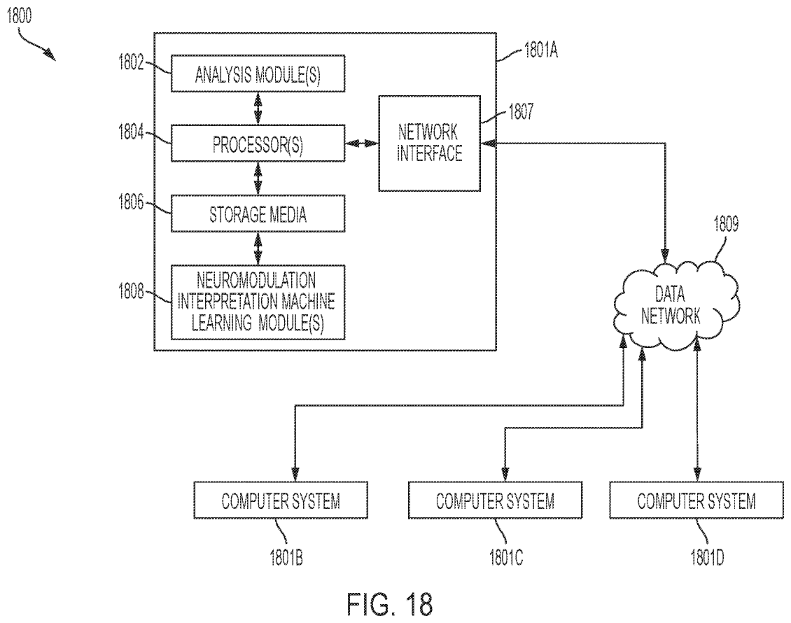

In some embodiments, any of the methods of the present disclosure may be executed by a computing system. FIG. 18 illustrates an example of such a computing system 1800, in accordance with some embodiments. The computing system 1800 may include a computer or computer system 1801A, which may be an individual computer system 1801A or an arrangement of distributed computer systems. The computer system 1801A includes one or more analysis module(s) 1802 configured to perform various tasks according to some embodiments, such as one or more methods disclosed herein. To perform these various tasks, the analysis module 1802 executes independently, or in coordination with, one or more processors 1804, which is (or are) connected to one or more storage media 1806. The processor(s) 1804 is (or are) also connected to a network interface 1807 to allow the computer system 1801A to communicate over a data network 1809 with one or more additional computer systems and/or computing systems, such as 1801B, 1801C, and/or 1801D (note that computer systems 1801B, 1801C and/or 1801D may or may not share the same architecture as computer system 1801A, and may be located in different physical locations, e.g., computer systems 1801A and 1801B may be located in a processing facility, while in communication with one or more computer systems such as 1801C and/or 1801D that are located in one or more data centers, and/or located in varying countries on different continents).

A processor can include a microprocessor, microcontroller, processor module or subsystem, programmable integrated circuit, programmable gate array, or another control or computing device.

The storage media 1806 can be implemented as one or more computer-readable or machine-readable storage media. The storage media 1806 can be connected to or coupled with a neuromodulation machine learning module(s) 1808. Note that while in the example embodiment of FIG. 18 storage media 1806 is depicted as within computer system 1801A, in some embodiments, storage media 1806 may be distributed within and/or across multiple internal and/or external enclosures of computing system 1801A and/or additional computing systems. Storage media 1806 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital video disks (DVDs), BLURAY® disks, or other types of optical storage, or other types of storage devices. Note that the instructions discussed above can be provided on one computer-readable or machine-readable storage medium, or alternatively, can be provided on multiple computer-readable or machine-readable storage media distributed in a large system having possibly plural nodes. Such computer-readable or machine-readable storage medium or media is (are) considered to be part of an article (or article of manufacture). An article or article of manufacture can refer to any manufactured single component or multiple components. The storage medium or media can be located either in the machine running the machine-readable instructions or located at a remote site from which machine-readable instructions can be downloaded over a network for execution.

It should be appreciated that computing system 1800 is only one example of a computing system, and that computing system 1800 may have more or fewer components than shown, may combine additional components not depicted in the example embodiment of FIG. 18, and/or computing system 1800 may have a different configuration or arrangement of the components depicted in FIG. 18. The various components shown in FIG. 18 may be implemented in hardware, software, or a combination of both hardware and software, including one or more signal processing and/or application specific integrated circuits.

Further, the steps in the processing methods described herein may be implemented by running one or more functional modules in an information processing apparatus such as general purpose processors or application specific chips, such as ASICs, FPGAs, PLDs, or other appropriate devices. These modules, combinations of these modules, and/or their combination with general hardware are all included within the scope of protection of the invention.

Neuromodulation and/or trend artifact removal interpretations, models and/or other interpretation aids may be refined in an iterative fashion; this concept is applicable to embodiments of the present methods discussed herein. This can include the use of feedback loops executed on an algorithmic basis, such as at a computing device (e.g., computing system 1800, FIG. 18), and/or through manual control by a user who may make determinations regarding whether a given step, action, template, model, or set of curves has become sufficiently accurate for the evaluation of the signal(s) under consideration.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. Moreover, the order in which the elements of the methods are illustrated and described may be re-arranged, and/or two or more elements may occur simultaneously. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A method, comprising:
receiving, from one or more electrodes, information identifying brain activity with a stimulus trend artifact signal for a first time period;
detecting, based on the information identifying the brain activity for the first time period, a trend in the stimulus trend artifact for the first time period and detrending the brain activity without the stimulus trend artifact signal for a second time period that is to occur after the first time period;
inserting the estimated brain activity without the stimulus trend artifact signal into the information for a second time period;
determining, based on the estimated brain activity for the second time period, a brain stimulus for the second time period; and
causing the brain stimulus to be applied the second time period.

2. The method of claim 1, wherein the brain stimulus is associated with a frequency and a phase determined based on the estimated brain activity for the second time period.

3. The method of claim 1, further comprising:
determining the stimulus trend artifact signal during the first time period associated with a prior brain stimulus; and
determining a trend artifact-removed brain activity for the first time period based on the trend artifact.

4. The method of claim 3, wherein estimating the brain activity for the second time period further comprises:
estimating the brain activity for the second time period based on the trend artifact-removed brain activity.

5. The method of claim 1, wherein the brain stimulus is caused to occur during a period of rhythmic brain activity.

6. The method of claim 1, wherein estimating the brain activity for the second time period further comprises:
estimating the brain activity using a parametric spectral estimation technique for modeling band limited oscillations.

7. A computer system, comprising:
a hardware processor;
a non-transitory computer readable medium that stores instructions that when executed by the hardware processor perform a method comprising:
receiving, from one or more electrodes, information identifying brain activity with a stimulus trend artifact signal for a first time period;
detecting, based on the information identifying the brain activity for the first time period, a trend in the stimulus trend artifact for the first time period and detrending the brain activity without the stimulus trend artifact signal for a second time period that is to occur after the first time period;
inserting the estimated brain activity without the stimulus trend artifact signal into the information for a second time period;
determining, based on the estimated brain activity for the second time period, a brain stimulus for the second time period; and
causing the brain stimulus to be applied the second time period.

8. The computer system of claim 7, wherein the brain stimulus is associated with a frequency and a phase determined based on the estimated brain activity for the second time period.

9. The computer system of claim 7, wherein the processor is configured to further perform the method comprising:
determining the stimulus trend artifact signal during the first time period associated with a prior brain stimulus; and
determining a trend artifact-removed brain activity for the first time period based on the trend artifact.

10. The computer system of claim 9, wherein estimating the brain activity for the second time period further comprises:
estimating the brain activity for the second time period based on the trend artifact-removed brain activity.

11. The computer system of claim 7, wherein the brain stimulus is caused to occur during a period of rhythmic brain activity.

12. The computer system of claim 7, wherein estimating the brain activity for the second time period further comprises:
estimating the brain activity using a parametric spectral estimation technique for modeling band limited oscillations.

13. A non-transitory computer readable medium that stores instructions that when executed by a hardware processor perform a method, comprising:
receiving, from one or more electrodes, information identifying brain activity with a stimulus trend artifact signal for a first time period;
detecting, based on the information identifying the brain activity for the first time period, a trend in the stimulus trend artifact for the first time period and detrending the brain activity without the stimulus trend artifact signal for a second time period that is to occur after the first time period;
inserting the estimated brain activity without the stimulus trend artifact signal into the information for a second time period;
determining, based on the estimated brain activity for the second time period, a brain stimulus for the second time period; and
causing the brain stimulus to be applied the second time period.

14. The non-transitory computer readable medium of claim 13, wherein the brain stimulus is associated with a frequency and a phase determined based on the estimated brain activity for the second time period.

15. The non-transitory computer readable medium of claim 13, further comprising:
determining the stimulus trend artifact signal during the first time period associated with a prior brain stimulus; and
determining a trend artifact-removed brain activity for the first time period based on the trend artifact.

16. The non-transitory computer readable medium of claim 15, wherein estimating the brain activity for the second time period further comprises:
estimating the brain activity for the second time period based on the trend artifact-removed brain activity.

17. The non-transitory computer readable medium of claim 13, wherein the brain stimulus is caused to occur during a period of rhythmic brain activity.

18. The non-transitory computer readable medium of claim 13, wherein estimating the brain activity for the second time period further comprises:
estimating the brain activity using a parametric spectral estimation technique for modeling band limited oscillations.
